(19) United States
(12) Patent Application Publication
(10) Pub. No.: US 2014/0114382 A1
(43) Pub. Date: Apr. 24, 2014

(54) STIMULATING BONE GROWTH AND CONTROLLING SPINAL CORD PAIN

(71) Applicant: KEUN-YOUNG ANTHONY KIM, IRVINE (KR)

(72) Inventor: KEUN-YOUNG ANTHONY KIM, IRVINE (KR)

(21) Appl. No.: 14/023,149

(22) Filed: Sep. 10, 2013

Related U.S. Application Data
(60) Provisional application No. 61/743,683, filed on Sep. 10, 2012.

(51) Int. Cl. A61N 1/32

(52) U.S. Cl. CPC A61N 1/326
USPC 607/116

(57) ABSTRACT

Bone growth for fusion promotion is stimulated in a mammalian patient in need thereof. Bone growth stimulation is achieved by implanting an electro-conductive bone growth stimulating implant in a region in the patient where bone growth is desired. An external device is worn by the patient to produce a direct current in the implant whereby bone growth is stimulated. The external device produces a magnetic field that induces an electric current in the implant. The electric current stimulates bone growth. The implant contains strips of a biocompatible conductive metal, such as, for example, nickel, gold or titanium. The strips can also be made of a conductive polymer such as for example, graphene. Implants to treat spinal cord pain are also disclosed.

### CROSS-REFERENCE TO RELATED APPLICATION

The present application claims priority under 35 U.S.C. §119(e) to U.S. Provisional Application No. 61/743,683, filed on Sep. 10, 2012, the disclosure of which is incorporated herein by reference in its entirety.

### FIELD OF THE INVENTION

The present invention relates to methods stimulating bone growth, methods of controlling pain and implants and devices to conduct said methods. In particular, implants containing electro-conductive strips are implanted in a mammalian patient in regions of the body to promote bone growth. An external device is used to produce an electric current along the electro-conductive material wherein the electric current promotes bone growth along the path of the current. Additionally, implants containing electro-conductive strips are implanted in a mammalian patient in regions adjacent to the spinal cord for pain control. An external device is used to produce an electric current along the electro-conductive strips wherein the electric current promotes pain relief.

### BACKGROUND OF THE INVENTION

It is estimated that about six million bone fractures, including about 600,000 non-union cases, occur annually in the United States, among which approximately 10% do not heal. In the orthopedic procedures conducted, about one million performed annually require allograft or autograft. One solution to enhancement of bone healing is through tissue engineering, in which cells, such as osteoblast, fibroblast, chondroblasts, are treated with bioactive signaling molecules, e.g., insulin or insulin mimetics or scaffolds such as β-TCP (tricalcium phosphate) and collagen under an appropriate environment. Current methods of treatment of bone fractures include (a) electro-stimulation devices (such as PEMF, Exogen and (b) biologics, such as bone morphogenic proteins (BMPs), e.g., rhBMP-2/ACS (INFUSE™ Bone Graft). The latter has been approved by FDA as an autograft replacement in spine fusion (ALIF) with specific interbody cages (2002), as an adjuvant for repair of tibia fractures with IM nail (2004), and for craniofacial maxillary surgery (2006), but this method is expensive, costing about $5,000 per application. (Lieberman, J. R., et al., J. Bone Joint Surg. Am., 2002, 84: 1032-1044; Trippel, S. B., et al., J. Bone Joint Surg. Am., 1996, 78: 1272-86.)

Fracture healing is a complex process that involves the sequential recruitment of cells and the specific temporal expression of factors essential for bone repair. The fracture healing process begins with the initial formation of a blood clot at the fracture site. Platelets and inflammatory cells within the clot release several factors that are important for chemotaxis, proliferation, angiogenesis and differentiation of mesenchymal cells into osteoblasts or chondroblasts.

The fracture healing process subsequent to the initial hematoma formation can be classified as primary or secondary fracture healing. Primary fracture healing occurs in the presence of rigid internal fixation with little to no interfragmentary strain resulting in direct bone formation across the fracture gap. Secondary fracture healing occurs in response to interfragmentary strain due to an absence of fixation or non-rigid fixation resulting in bone formation through intramembranous and endochondral ossification characterized by responses from the periosteum and external soft tissue.

Intramembranous bone formation originates in the periosteum. Osteoblasts located within this area produce bone matrix and synthesize growth factors, which recruit additional cells to the site. Soon after the initiation of intramembranous ossification, the granulation tissue directly adjacent to the fracture site is replaced by cartilage leading to endochondral bone formation. The cartilage temporarily bridging the fracture gap is produced by differentiation of mesenchymal cells into chondrocytes. The cartilaginous callus begins with proliferative chondrocytes and eventually becomes dominated by hypertrophic chondrocytes. Hypertrophic chondrocytes initiate angiogenesis and the resulting vasculature provides a conduit for the recruitment of osteoblastic progenitors as well as chondroclasts and osteoclasts to resorb the calcified tissue. The osteoblastic progenitors differentiate into osteoblasts and produce woven bone, thereby forming a united fracture. The final stages of fracture healing are characterized by remodeling of woven bone to form a structure, which resembles the original tissue and has the mechanical integrity of unfractured bone.

The processes of bone metabolism vary from bone repair. Bone metabolism is the interplay between bone formation and bone resorption. Bone repair, as described above, is a complex process that involves the sequential recruitment and the differentiation of mesenchymal cells towards the appropriate osteoblastic/chondrogenic lineage to repair the fracture/defect site.

Fractures, or broken bones, are common injuries that can take months or even years to fully heal. The healing process is generally the same for all fractures. Through a series of stages, new bone forms and fills in the fractured area. The rate of healing and the ability to remodel a fractured bone vary tremendously for each person and, in general, depend on several factors, such as age, overall state of health, the type of fracture, and the bone involved. Specifically, smoking, diabetes, obesity, and advanced age can increase the difficulty of fracture healing due in part to diminished circulation, and other factors not well understood. Complications of orthopedic surgery and trauma include non-union or poor union of fractures at fusion sites. Despite improvement in fusion-promoting devices and chemicals, accelerated and complete healing and fusion between bone surfaces remains at times elusive.

The use of electrical stimulation to improve the effectiveness of fracture healing has grown significantly in recent years. Electrical or ultrasound stimulation is a good option for patients who have bone healing problems, or fractures that have poor healing potential. As the number of scientific and clinical studies validating the use of electrical or ultrasound stimulation to enhance spine fusion has increased, there is a better understanding among spine surgeons about how and when to use specific electrical stimulation devices to aid in the healing of spine fusion. Some of the problems associated with this type of treatment include patient compliance and accuracy in the placement of the simulator. Typical treatment regimens include applying the bone growth stimulator to the fracture for about 20 minutes to up to 4 hours per day in order to provide a benefit. In addition, the placement of the stimulators must be such that the bone is sufficiently stimulated. Despite the improved understanding of micro-vibration and micro-electric potentials generated in situ by bone to promote bone healing during standard or typical stress (Wolff's law), it is still unclear how one may harness and augment the endogenous bioelectric potentials to promote further bone growth. Implantable electric bone growth stimulators require an additional surgery for removal of the device which is always a downside especially for the elderly.

In patients with bone trauma and/or advanced spinal degeneration fusion remains the goal. Two bone surfaces are required to form a healing callous of bone that connects the two in order to strengthen a fracture or an abnormal motion segment such as seen in spondylolisthesis of the spine. Instrumentation, such as, pedicle screws and rods, and biochemical technology, such as, bone morphogenic proteins, have been utilized to attempt this fusion. The shortcomings of these technologies are potentially extreme. Bone morphogenic protein is alleged to produce cancer and male sterility and has shown to produce cyst-like abnormal bone growth and soft tissue swelling. Zara, et al, Tissue Eng. Part A. 2011, May, 17 (9-10): 1389-1399. Pedicle screws are notoriously associated with non-union or pseudoarthrosis rates. With the aging population operations to fix broken bones and non-healing callouses in patients with osteoporosis is a growing problem. The ability of piezoelectric pulses, ultrasound, direct currents and inductive coupling have shown promise in forming new bone in turkey and rabbit models as well as in humans. Certain braces create an electromagnetic field around the wearer in order to promote inductive coupling, ie, generate a current in situ, with questionable results.

There is currently no orthopedic implant that (a) augments an external electromagnetic field internally into a direct current and (b) attempts to use conductive properties of an internal metal alloy and chemicals to induce a current from one bone surface to another during mechanical stress. The present invention provides both of these concepts to improve fusion healing in non-union bone fractures. The present invention is especially useful in promoting osteogenesis in high risk patients, such as, smokers, diabetics, the elderly, patients with osteoporosis to name a few.

### SUMMARY OF THE INVENTION

Briefly, in accordance with the present invention, bone growth for fusion promotion is stimulated in a mammalian patient in need thereof. Bone growth stimulation is achieved by implanting an electro-conductive bone growth stimulating implant in a region in the patient where bone growth is desired. An external device is worn by the patient to produce a direct current in the implant whereby bone growth is stimulated. The external device produces a magnetic field that induces an electric current in the implant. The electric current stimulates bone growth. Bone growth can be stimulated in any mammal, including but not limited to, a human, a dog, a cat, an agricultural mammal or a horse. The implant contains strips of a biocompatible conductive metal, such as, for example, nickel, gold or titanium. The strips can also be made of a biocompatible conductive polymer such as, for example, graphene.

Additionally, the present invention relates to managing pain or pain reduction in patients with spinal cord pain. Pain relief is achieved by implanting an electro-conductive implant in a region adjacent to the spinal cord where pain relief is needed. An external device is worn by the patient to produce a direct current in the implant whereby pain is reduced. The external device produces a magnetic field that induces an electric current in the implant. The electric current acts as a spinal cord stimulator to manage pain. Pain relief can be stimulated in any mammal including, but not limited to, a human, a dog, a cat, an agricultural mammal or a horse. The implant contains strips of a biocompatible conductive metal, such as, for example, nickel, gold or titanium. The strips can also be made of a biocompatible conductive polymer, such as, for example, graphene.

Of particular interest in practicing the present invention, a biomechanical spacer or cage is lined with strips of gold or other biocompatible conductive metal or polymer. The gold is positioned from top to bottom of the spacer and, when activated by a magnetic field, will produce a direct electric current from one side of a fractured bone to the other side of the fracture thereby stimulating bone growth across the fractured zone and thereby reducing the incidence of non-union healing. The direct electric current is created by the patient wearing an external device, such as, for example, a brace, a belt, a corset, a strap or a band that produces an electric field adjacent to or around the site of the implant. The electric field interacts with the gold strips to produce a current that promotes bone growth.

The present invention provides implants and methods that result in improved healing of fractured bones and promotes fusion of bone fractures. Because the present implants do not contain batteries, surgical removal of the implant is unnecessary. Additionally, patients at a high risk for non-union healing have an improved recovery and a higher success rate for complete bone fusion.

### BRIEF DESCRIPTION OF THE FIGURES

FIGS. 1A and 1B show a biomechanical spacer that contains biocompatible electro-conductive strips.

FIG. 2 shows a representation of a broken bone treated with a bone stimulating implant.

### DETAILED DESCRIPTION OF THE INVENTION

When used herein the following terms have definitions described below:

The term “mammal” when used herein includes any mammal especially humans. Non-human mammals include non-human primates, zoo animals, performance mammals, such as, race horse and breeding animals, and companion animals such as dogs and cats.

The term “strip(s)” when used herein refers to an electro-conductive material; “material” means strands, filaments, elongated pieces of foil and wires of electro-conductive material including any narrow elongated configuration of said material(s).

In practicing the present invention, bone growth for fusion promotion is stimulated in a mammalian patient. The bone fusion treats a bone fracture, which includes bone degeneration from osteoporosis such as is needed in a spinal fusion. Bone growth stimulation is achieved by implanting an electro-conductive bone growth stimulating implant in a region in the patient where bone growth is desired. Preferably, the implant contains strips of electro-conductive materials (conductive metals, conductive polymers) that are positioned along the length of the implant. The implant can be placed between the bone surfaces to be fused, onto, or near, hardware (biomechanical spacers (cages), screws and rods) or in the region where bone growth is desired. Once an electro-conductive bone growth stimulating implant is in place, an external device is worn by the patient to produce a direct current in the implant whereby bone growth is stimulated. The direction of bone cell growth and migration will follow the direction of the electro-conductive material in the implant. An external device is worn by the patient around the area of the implant to produce a magnetic field that induces an electric current in the implant's electro-conductive strips. The electric current stimulates bone growth. Bone growth can be stimulated in any mammal, including, but not limited to, a human, a dog, a cat, an agricultural mammal or a horse.

The implant contains strips of a biocompatible electro-conductive metal, such as, for example, nickel, gold, a suitable metal alloy or titanium. The strips can also be made of a conductive polymer, such as, for example, graphene. The exact shape and size of the strips are not critical to the practice of the present invention. The strips can be foil strips or small diameter wire or filaments. The strips are preferably arranged in the implant so as to linearly connect a first bone surface with a second bone surface where the two surfaces are desired to be fused to heal a bone fracture or fuse spinal vertebrae. Strips are usually about 0.1 mm to about 10 mm in diameter and preferably from about 1-2 mm. When foil metallic conductors are used the foil can be 0.1 mm to about 1.0 mm thick and have a width of from about 0.1 mm to about 10 mm. Preferably the gold foil is about 0.127 mm thick and from 1-2 mm in width.

The implant of the present invention contains a biocompatible substrate wherein the electro-conductive materials or strips are affixed to, or embedded in, the substrate. Suitable substrates include hardware such as biomechanical spacers (cages), screws and rods. Substrates also include osteoconductive scaffolding materials that promote bone growth such as autografts, allografts and synthetic osteoconductive scaffolds such as hypoxyapetite and β-tricalcium phosphate. The substrates can optionally contain piezoelectric crystals.

The present implants can be pre-made by manufacturers who supply surgical hardware and osteoconductive scaffolding materials by incorporating biocompatible electro-conductive strips into their products as described herein, ie, by making sure that the strips run in a direction across the fracture in order to promote complete bone fusion and reduce the chance of non-union healing. Alternatively, the present implants can be in the surgical suite as a patient is being operated on for a bone fracture or spinal fusion. The electro-conductive materials are added to a substrate in the surgery suite as a bone fracture surgery or spinal surgery is being conducted. For example, the surgery team can line the hollow portion of a spacer with gold filaments and then add an osteoconductive scaffolding material into the hollow portion which can additionally hold the strips in place.

Any biocompatible material can be used to form all or part of a spacer that will serve as the substrate of the present implant. Suitable materials include, titanium, stainless steel and/or other surgical grade metals and metal alloys. In addition, various polymers, such as polyetheretherketone (PEEK), can also be used to form at least part of the spacer implant. The electro-conductive strips are preferably used to line the inside of the cage in a vertical arrangement from top to bottom. The number of vertical strips is not critical and can range from 1-100 or more but preferably a plurality of strips are employed on all sides of the spacer.

In another embodiment of the present invention, an implant is made by incorporating electro-conductive strips into an osteoconductive scaffolding material that is placed in the junction between the two bones that are to be fused. The strips are positioned to run from a first bone surface to a second bone surface. In a preferred embodiment, β-tricalcium phosphate is used as an osteoconductive material that has incorporated into it an electro-conductive material such as gold filaments.

The external device worn by the patient produces a direct current in the strips contained in the implant whereby bone growth is stimulated. The external device can be any brace, belt, harness, corset, strap or band that surrounds the implant and can be worn by the patient. The external device can contain magnets or electric coils with a power supply to provide a current. The external device emits an electro-magnetic field, preferably variable, which according to Faraday's law will generate an electric pulse in the center of the field thereby resulting in a direct current being imparted to the strips in the implant. The direct current stimulates bone growth. In one embodiment the external emitter produces an electromagnetic field varying from 0.1 to 20 G to create an electrical field at the fracture site of 1 to 100 mV/cm. Griffin, et al, Electrical Stimulation in Bone Healing: Critical Analysis by Evaluating Levels of Evidence, ePlasty, Vol. 11, July 26, 2011, p. 303-353.

In another embodiment of the present invention, a spacer cage used for anterior lumbar interbody surgery or anterior cervical interbody surgery according to the present invention is used to stimulate bone growth and promote fusion. In a further embodiment the spacer cage contains electro-conductive materials (gold, zinc, titanium, etc) at the ends of the cage that generate small electric currents with micro-motion. Each compressive motion will generate a micro-current or piezioelectric current to further promote fusion.

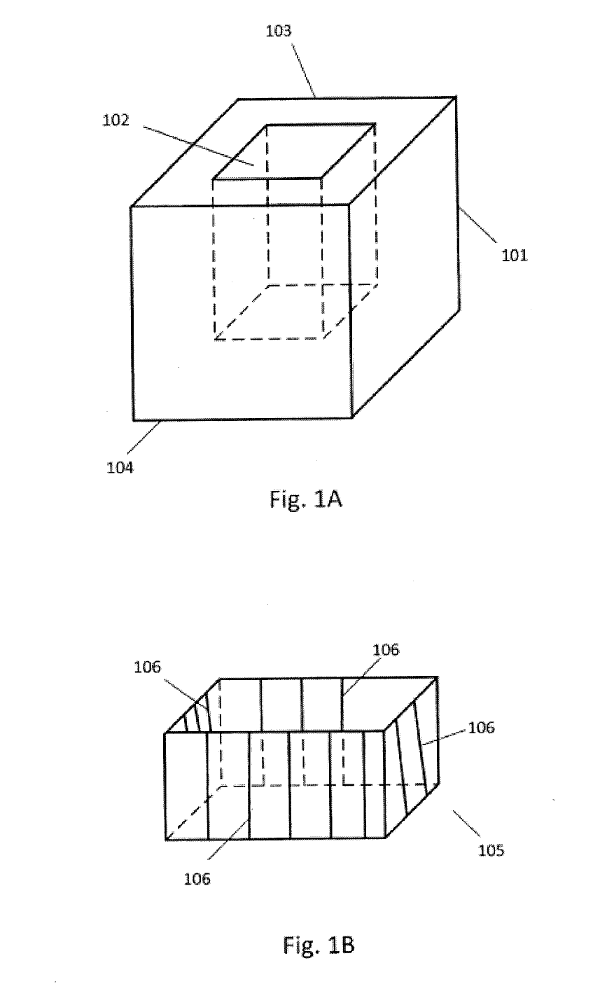

Referring to the drawings, FIG. 1A shows a perspective view of a biomechanical spacer 101 implant of the present invention containing a hollowed out interior 102 and two bone contact surfaces 103, 104. Bone contact surface 103 abuts against a first bone surface (not shown) and bone surface 104 abuts against a second bone surface (not shown). FIG. 1B shows a cutout view 105 of the interior 102 showing electro-conductive strips 106 that run vertically from the first bone surface (not shown) to the second bone surface (not shown). Implant 101 is implanted in a mammal between two bone surfaces resulting from trauma (broken bone) and when the patient wears an external device (not shown) around the body adjacent to where the implant is located it produces an electromagnetic field and a current is created in the electro-conductive strips 106 thereby stimulating bone formation resulting in a fully healed union between the first bone surface 103 and second bone surface 104.

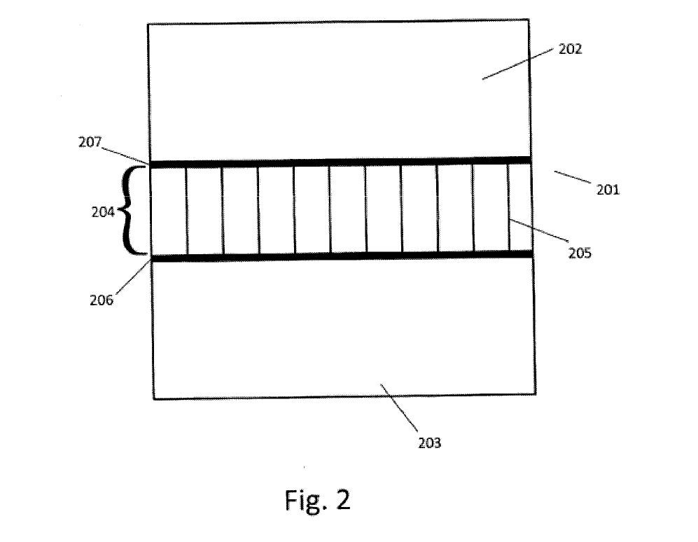

FIG. 2 shows a cross sectional view of a bone fracture 201 that has a proximal bone section 202, a distal bone section 203 and an implant cage of the present invention 204. Cage 204 contains a plurality of electro-conductive strips 205 running from the proximal bone section 202 to the distal section 203. The ends of electro-conductive strips 205 come into close proximity to the distal end bone surface 206 and proximal end bone surface 207. When the patient wears an external device (not shown) around the body adjacent to where the implant is located the device produces an electromagnetic field and a current is created in the electro-conductive strips 205 thereby stimulating bone formation resulting in a fully healed union between the distal bone section 203 and the proximal bone section 202.

Another aspect of the present invention relates to a method of reducing spinal cord pain in a mammalian patient by implanting an electro-conductive implant in a region in the patient directing electric current in the implant whereby pain is reduced. In this regard the implant acts as a spinal cord stimulator without the need for lead wires or batteries. In this embodiment the implant contains strips of a biocompatible conductive metal or conductive polymer as described above with respect to the present implants used to promote bone fusion and bone growth. In this application for spinal cord stimulation the implant is made of a biocompatible substrate and the strips of electro-conductive material so as to fit the anatomy of the spine. The implant is positioned in a surgical procedure at a location adjacent to where the spinal cord pain occurs. An external device is worn by the patient wherein the device surrounds the area of the implant and produces a magnetic field that creates a direct current in the implant. The direct current reduces pain similarly to a traditional spinal cord stimulator. The biocompatible electro-conductive metal is gold, nickel or titanium. The spinal cord stimulation according to the present invention is used for pain relief, nerve regeneration, and ischemic foot or leg syndrome.

A bone growth inhibitor can optionally be added to the biocompatible substrate in an implant used for spinal cord stimulation to prevent unwanted bone growth in the region where the implant is located. Bone growth inhibitors include nerve growth factor (NGF) and PEEK.

In one embodiment of the present invention for use as a spinal cord stimulator, the implant comprises a calcium phosphate substrate, preferably β-tricalcium phosphate, and strips of gold, nickel or titanium that are fixed or embedded into the calcium phosphate substrate. A preferred electro-conductive material is gold.

The following example illustrates the practice of the present invention but should not be construed as limiting its scope.

### EXAMPLE 1

### Femur Fusion

A human patient presents with a broken femur. A mechanical spacer/cage shown in FIGS. 1A and 1B is surgically implanted between the proximal and distal femur so that the cage abuts the distal femur and the proximal femur. The interior of the cage contains a plurality of gold strips that run from the proximal femur to the distal femur and an osteoconductive scaffolding material such as autologous bone. The patient is given a leg band or wrap to wear around the femur adjacent to where the implant is located. The leg band/wrap emits an electromagnetic field which produces a current in the gold strips resulting in bone formation and resulting in a fully healed union between the distal femur and the proximal femur.

Additional surgical procedures are performed using the implants of the present invention to repair non-union long bone fractures. Non-union podiatry fractures, non-union spinal fractures and skull fractures with cranioplasty.

The present invention can additionally be described as:
1. A method of reducing spinal cord pain in a mammalian patient in need thereof which comprises:
   a. implanting an electro-conductive implant in a region in the patient where pain reduction is desired;
   b. providing outside the patient's body a device that produces a direct electric current in the implant whereby pain is reduced.
2. The method of 1 above wherein the implant contains strips of a biocompatible conductive metal or conductive polymer and the device produces a magnetic field.
3. The method of 2 above wherein the biocompatible conductive metal is gold, nickel or titanium.
4. The method of 4 above wherein the device produces a magnetic field that produces an electric current in the gold, nickel or titanium strips.
5. A spinal cord stimulating implant which comprises:
   a. a calcium phosphate substrate and
   b. strips of gold, nickel or titanium fixed in the calcium phosphate.
6. In an implant for promoting bone growth at a bone fracture site containing a first bone surface and a second bone surface, the improvement which comprises:
   a plurality of strips of an electro-conductive material positioned in the implant from the first bone surface to the second bone surface.
7. The improved implant of 6 above wherein the electro-conductive material is gold, nickel or titanium.
8. In a method for promoting bone growth at a bone fracture site containing a first bone surface and a second bone surface, the improvement which comprises:
   a. implanting an electro-conductive bone growth stimulating implant in between the first bone surface and the second bone surface wherein the implant contains a plurality of strips of an electro-conductive material positioned in the implant from the first bone surface to the second bone surface, and;
   b. providing outside the patient's body, a device that produces a direct current in the electro-conductive material whereby bone growth is stimulated.
9. The improved implant of 8 above wherein the electro-conductive material is gold, nickel or titanium and the device emits a magnetic field.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

All patents, published patent application, references and publications cited above are incorporated herein by reference.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

All patents, published patent application, references and publications cited above are incorporated herein by reference.

I claim:

1. A method of stimulating bone growth for fusion promotion in a mammalian patient in need thereof which comprises:
a. implanting an electro-conductive bone growth stimulating implant in a region in the patient where bone growth is desired;
b. providing outside the patient's body a device that produces a direct current in the implant whereby bone growth is stimulated.

2. The method of claim 1 wherein the implant contains strips of a biocompatible conductive metal or conductive polymer and the device produces a magnetic field and the device emits a magnetic field.

3. The method of claim 2 wherein the biocompatible conductive metal is gold, nickel or titanium.

4. A method of stimulating bone growth for fusion promotion in a mammalian patient which comprises:
a. providing an electro-conductive biomechanical spacer/cage that contains a plurality of electro-conductive strips wherein said conductive strips are configured in a spatial arrangement to promote bone growth in a desired direction;
a. implanting said spacer/cage in a region in the patient where bone growth is desired;
b. providing outside the patient's body a device that produces an direct electric current in the conductive strips whereby bone growth is stimulated.

5. The method of claim 4 wherein the implant contains strips of a biocompatible conductive metal or conductive polymer and the device produces a magnetic field.

6. The method of claim 5 wherein the biocompatible conductive metal is gold, nickel or titanium.

7. A bone growth kit which comprises:
a. an electro-conductive bone growth stimulating implant and
b. an external device that is capable of creating a direct electric current in the implant wherein the device is worn by a mammalian patient.

8. The kit of claim 7 wherein implant contains strips of a biocompatible conductive metal or conductive polymer and the external device emits a magnetic field.

9. The kit of claim 8 wherein the biocompatible conductive metal is gold, nickel or titanium.

10. An electro conductive mammalian implant to induce fusion promotion of bone which comprises:
a. a biocompatible substrate and
b. an electro-conducting material that produces a direct electric current when stimulated from a source outside the mammal.

11. The implant of claim 10 wherein the substrate is an autograft, an allograft or a synthetic osteoconductive scaffold.

12. The implant of claim 11 wherein the electro-conductive material comprises strips of a biocompatible conductive metal or conductive polymer and the direct electric current is produced in said strips of conductive metal and conductive polymer by subjecting them to a magnetic field.

13. The implant of claim 12 wherein the biocompatible conductive metal is gold, nickel or titanium.

14. An electro conductive mammalian implant to induce fusion promotion of bone which comprises:
a. an osteoconductive scaffolding, and
b. strips of biocompatible electro-conductive material oriented in a linear direction of desired bone growth.

15. The implant of claim 14 wherein the biocompatible conductive metal is gold, nickel or titanium.

16. The implant of claim 15 wherein the osteoconductive scaffolding is an autograft, an allograft or a synthetic osteoconductive scaffold.

17. A method of stimulating bone growth for fusing a first bone surface to a second bone surface in a mammalian patient in need thereof which comprises:
a. implanting an electro-conductive bone growth stimulating implant between the first bone surface and the second bone surface;
b. providing outside the patient's body a device that produces a direct current in the implant whereby bone growth is stimulated.

18. The method of claim 17 wherein the implant contains a plurality of strips of an electro-conductive material positioned in the implant in substantially a linear fashion from the first bone surface to the second bone surface and the device emits a magnetic field.

19. The method of claim 18 wherein the electro-conductive material is gold, zinc or titanium.
